(19) United States
(12) Patent Application Publication
(10) Pub. No.: US 2002/0054921 A1
(43) Pub. Date: May 9, 2002

(54) Use of phyllanthus components for the treatment or prophylaxis of infections triggered by flavivirdae

(76) Inventors: Christoph Von Keudell, Ammerland (DE); Robert H.M. van der Giessen, Aesch (CH)

Correspondence Address: BAKER BOTTS LLP, C/O INTELLECTUAL PROPERTY DEPARTMENT, THE WARNER, SUITE 1300, 1299 PENNSYLVANIA AVE, NW, WASHINGTON, DC, 20004-2400, US

(21) Appl. No.: 09/884,461

(22) Filed: Jun. 20, 2001

(30) Foreign Application Priority Data
Jun. 20, 2000 (DE) 100 30 139.8

(51) Int. Cl. A61K 35/78; A01N 25/00

(52) U.S. Cl. 424/725; 424/405

(57) ABSTRACT

The invention relates to the use of one or more Phyllanthus components or substances or mixtures of substances obtained therefrom for preventing or treating infectious diseases that are triggered by viruses belonging to the family of Flaviviridae or infectious diseases in which viruses belonging to the family of Flaviviridae are involved in the development or progression thereof. Furthermore, the invention relates to the use of one or more Phyllanthus components or substances or mixtures of substances obtained therefrom for producing a pharmaceutical composition for the prevention or treatment of infectious diseases that are triggered by viruses belonging to the family of Flaviviridae or infectious diseases in which viruses belonging to the family of Flaviviridae are involved in the development or progression thereof. The invention also relates to the use of one or more Phyllanthus components or substances or mixtures of substances obtained therefrom for inhibiting the propagation of viruses belonging to the family of Flaviviridae. Moreover, the invention relates to a method for preventing or treating infectious diseases in a mammal that are triggered by viruses belonging to the family of Flaviviridae or infectious diseases in which viruses belonging to the family of Flaviviridae are involved in the development or progression thereof, wherein one or more Phyllanthus components or substances or mixtures of substances obtained therefrom are administered to the mammal. In addition, the invention relates to a method for inhibiting the propagation of viruses belonging to the family of Flaviviridae, wherein one or more Phyllanthus components or substances or mixtures of substances obtained therefrom are contacted with the viruses. The hepatitis C virus is a preferred virus.

The invention relates to the use of one or more Phyllanthus components or substances or mixtures of substances obtained therefrom for preventing or treating infectious diseases that are triggered by viruses belonging to the family of Flaviviridae or infectious diseases in which viruses belonging to the family of Flaviviridae are involved in the development or progression thereof. Furthermore, the invention relates to the use of one or more Phyllanthus components or substances or mixtures of substances obtained therefrom for producing a pharmaceutical composition for the prevention or treatment of infectious diseases that are triggered by viruses belonging to the family of Flaviviridae or of infectious diseases in which viruses belonging to the family of Flaviviridae are involved in the development or progression thereof. The invention also relates to the use of one or more Phyllanthus components or substances or mixtures of substances obtained therefrom for inhibiting the propagation of viruses belonging to the family of Flaviviridae. Moreover, the invention relates to a method for preventing or treating infectious diseases in a mammal that are triggered by viruses belonging to the family of Flaviviridae or of infectious diseases in which viruses belonging to the family of Flaviviridae are involved in the development or progression thereof, wherein one or more Phyllanthus components or substances or mixtures of substances obtained therefrom are administered to the mammal. In addition, the invention relates to a method for inhibiting the propagation of viruses belonging to the family of Flaviviridae, wherein one or more Phyllanthus components or substances or mixtures of substances obtained therefrom are contacted with the viruses. The hepatitis C virus is a preferred virus.

Several documents are cited throughout the text of this specification either by name or are referred to by numerals to within parenthesis. The disclosure content of each of the documents cited herein (including any manufacturer's specifications, instructions, etc.) is herewith incorporated herein by reference.

The plant genus Phyllanthus belongs to the subfamily of Phyllantoideae which belong to the family of Euphoribiaceae. The genus Phyllanthus comprises approximately 700 species altogether which grow in the tropical and subtropical regions in Australia, China, the Philippines, Thailand, Indonesia, Burma, India, East and West Africa, North America, Mexico, Cuba, the Caribbean and Venezuela. Only very rarely are there members of the genus Phyllanthus in the northern temperate zones.

Due to the heterogeneity of the genus Phyllanthus only very few genus-specific features are known. Since it is not possible to draw an exact line between the individual genera and since some Phyllanthus species may be assigned to different groups, depending on one's personal point of view, it is very difficult to get a clear picture of substances of groups of substances that are sufficiently specific to the genus. The only feature they all have in common is the lack of milk, which is a family feature of the subfamily Phyllanthoideae. However, some characteristic groups of substances are present in Phyllanthus to a more or less large extent, with the alkaloids, in particular the alkaloids of the prevalent securinine type, which are only present in some species being an example thereof. Alkaloids, for instance, were detected in all the parts of a plant of Phyllanthus niruri. Furthermore, there rarely are compounds belonging to the cyanogenic glycosides. They belong to the descendants of the class that are derived from tyrosine, a typical member thereof being taxiphyllin and 2-hydroxy-2-(4-hydroxyphenyl)acetone nitrile in the leaves of Phyllanthus gasstroemii. Tanning agents (tannines), in particular, gallotannins and ellagitannins as well as their precursors are preferably present in woody species in the tropics. The main members are Phyllanthus emblica having phyllemblin (ethyl gallate) in their fruits. In addition, phyllanthusin, a derivative of ellagitannin, in the herb of Phyllanthus amarus has been described. Another large group of substances relates to the lignans, with lignans such as phyllanthin and cyclolignans or tetralignans like hypophyllanthin being found in the herb of Phyllanthus amarus and in all parts of Phyllanthus niruri. Moreover, sesquiterpenes in Phyllanthus acuminatus have been described which chemically belong to the epoxides of tricyclic sesquiterpene esters having a spirane structure and to the bioses bound in part like ester. In addition, triterpenes are comparatively abundant in all parts of the plants. Apart from phytosterols (sitosterol), descendants of the friedelan, olean and lupan type (betula camphor and glochidonol in Phyllanthus reticulatus), the presence of the euphan type is striking such as phyllanthenol in Phyllanthus niruri.

In particular, Phyllanthus amarus and Phyllanthus niruri are used in folk medicine for treating fever, e.g. in Nigeria, Cuba or Jamaica (Unander et al., 1991, J. Ethno Pharmacol. 34: 97-133). Apart from that, the herbs of several Phyllanthus species is used for treating diarrhea, for increasing the amount of urine, as a laxative and in the case of spasms and colics. Furthermore, the effectiveness of Phyllanthus for treating diabetes (Unander, loc. cit.) have been described, however, the effectiveness in theses forms of application has not been scientifically proven.

In scientific tests, it was possible to show that a plurality of hydrolyzable tannins of the genus Phyllanthus inhibit important eukaryotic protein kinases such as the cAMP-dependent protein kinase of the rat liver, the Ca²⁺-dependent protein kinase of the wheat embryo, the Ca²⁺ and phospholipid-dependent protein kinase C (PKC) of the rat brain, wherein the tannins amariin, geraiin and the phenazine derivative of geraiin are most effective. Moreover, it has been scientifically proven that the dry extract of Phyllanthus amarus, which had been extracted with 95% ethanol for three hours under reflux from the plant, had an anti-microbial effect and inhibited the growth of Bacillus subtilis and Staphylococcus aureus. In an agar plate diffusion-test in which 0.1 ml of such an aqueous extract was added at a concentration of 50 mg/ml, a moderate inhibitory effect (inhibition zone<15 mm). A positive control of 0.1 ml streptomycin at a concentration of 10 mg/ml resulted in inhibition zone of 35 mm. In the same test system, the extract was ineffective against Pseudomonas aeruginosa, Escherichia coli, Aspergillus niger and Candida albicans. In contrast, a dry extract of the whole plant which had been extracted with methanol at 20° C. was effective against Staphylococcus aureus, but not against E. coli, A. niger and C. albicans.

Furthermore, an anti-viral effect of Phyllanthus against the hepatitis B virus (HBV) is known. An aqueous extract of the dried and powdered drug (40 g in 200 ml water, extracted for two hours at 60° C.) inhibits the binding of HbsAg antibodies to the surface antigen of HBV (HbsAg) in vitro and inhibits the viral DNA-polymerase of the HBV and the hepatitis virus of the groundhog.

Apart from HBV, the hepatitis A virus and the hepatitis C virus (HCV) are also known as viruses triggering hepatitis, yet, they all belong to different virus families. The only feature these viruses have in common is their replication in human parenchymal liver cells (human hepatocytes) which are the primary target cells of these viruses.

HCV belongs to the family of Flaviviridae which has three genera (flavivirus, pestivirus, HCV and hepatitis G virus (HGV)). Apart from the yellow fever virus, the genus flaviviruses also include a number of viruses being pathogenic for humans which may be transmitted by insects and Arachnida. Some could be connected with feverish hemorrhagic diseases and encephalites in tropical countries which correspond to the zone in which specific gnats transmit the virus. These include, amongst others, the dengue fever and, in particular in Central Europe, the pathogen of the Central European tick-borne fever which is transmitted by tick bites and which is endemic in certain regions. The second genus comprises the pestiviruses which trigger grave animal epidemics such as the classic hog cholera triggered by the hog cholera virus. However, these viruses are not transmitted by insects.

For a long time, the hepatitis C virus was assigned to the so-called nonA/nonB hepatitis viruses. It was only in 1999, that the genome of these viruses was characterised. Today, 12 different genotypes of the HCV from different geographic regions are known. In the early 90s, HCV was assigned to the family of Flaviviridae as a proper genus due to its molecular biological characteristics. Before suitable test methods were introduced, HCV caused 80-90% of all nonA/nonB hepatites. HCV is only found in humans and, most of the time, it was transmitted via blood transfusions or blood products. One in 3000-5000 blood donations is positive, with the risk of being infected when receiving positive banked blood being approximately 75%. Hepatitis transmitted via transfusion often is chronic. Further possibilities of transmission include common use of injections and injection tubes by drug addicts and, to a significantly smaller extent, sexual contact with infected persons. It is much more rarely that hepatitis is transmitted via contacts with patients living in the same house due to insufficient hygienic conditions. In general, hospital staff is to be regarded as a very endangered group of people. Moreover, it is known that during pregnancy a mother can transmit the virus to her baby. In spite of good diagnostic examination possibilities, the source of infection is not exactly known in a plurality of cases of infection.

With a number of HCV infections, there is an inflammation of the liver after an incubation time of 6 to 8 weeks, the course of which generally is not very complicated. About 75% of the infections have a clinically inapparent course and a complicated course is very rare. Approximately up to 80% of all infected suffer from chronically persistent or chronically reactive hepatites. In the blood of these patients viruses can be detected. The chronic infection are characterised by an increased level of transaminase. In 3-20% of the chronic cases, a cirrhosis is formed, about 4% of these patients suffer from a primary hepatocellular carcinoma. The liver is the target organ of the HCV, with the virus being transported to the liver via infected macrophages. There it infects the human hepatocytes and destroys them, resulting in an inflammation of the liver with cell necroses. The hepatocytes produce and secrete interferon-α. With an electron microscope tubular structures can be seen in the cytoplasma of the infected hepatocytes. However, up to know only little has been known as to the details of the pathogenesis of the acute infection. In the case of chronic infections, antigen-antibody complexes are formed which may be deposited in the glomerula. These complexes are considered to be responsible for the membranoproliferative glomerulonephritis with these patients.

HCV has a high rate of mutation and changes in the course of an infection in patients. This is why it is called a new quasi-species. The probability of the base changes occurring during the replication is 2×10³. It is due to the fact that, in contrast to the cellular DNA polymerases, the RNA-dependent RNA polymerase of the virus cannot check the reading accuracy. As already mentioned, the HCV belongs to the group of flaviviruses the infectious particles of which have a diameter of approx. 40-50 mm. The capsids, which only consist of one viral protein (C-protein), are surrounded by a coat membrane in which two viral surface proteins are deposited. The inside of the capsids contains the RNA genome which closely interacts with the strongly basic C-protein. The genome of the flaviviruses consists of a single-stranded RNA and has an overall length of approx. 9100-11000 bases, with HCV having approx. 9500 bases. The RNA is present in positive-strand orientation and is characterised by a single, large, continued open reading frame encoding a polyprotein which is proteolytically cleaved into the different protein components. Both enzymes which, as parts of the polyprotein, have a autocatalytic effect and which are activated and the cellular signalases are responsible therefor. Compared to the two other genera flaviviruses and pestiviruses, the genome of the HCV is different. With a length of 340 bases, the non-translated region at the 5′ end is significantly longer and exerts the function of an IRES (internal ribosomal entry site), which mediates the binding of ribosome subunits and which is necessary for the regulated translation of the polyprotein. The most important individual proteins of the HCV in comparison with other 20 members of flaviviruses (yellow fever virus and Central European tick-borne fever virus) are listed in the table below.

TABLE 1
Comparison and function of the flavivirus-specific proteins

| | | Central | | |
|---|---|---|---|---|
| | | European | | |
| | | tick-borne | | |
| | yellow fever | fever | hepatitis C | |
| protein | virus | virus | virus | Function |
| C | 12-14 kD | 13-16 kD | 22 kD | capsid protein interaction |
| | | | | with the RNA genome |
| M | 7-9 kD | 7-8 kD | — | Membrane protein |
| E | 51-59 kD | 50-60 kD | — | Membrane protein |
| | | | | glycosylated, neutralised |
| | | | | antibodies, hemagglut- |
| | | | | inin, adsorption |
| E1 | — | — | 31-35 kD | Membrane protein |
| | | | | glycosylated |
| E2 | — | — | 68-72 kD | Membrane protein |
| | | | | glycosylated |
| NS1 | 19-25 kD | 39-41 kD | — | Glycosylated, cell |
| | | | | membrane-associated, |
| | | | | secreted, no |
| | | | | component of the virus |
| | | | | particles |
| NS2A | 20-24 kD | 20 kD | 6 kD | ? |
| NS2B | — | 14 kD | 23 kD | Zn²-metalloproteinase, |
| | | | | associated with NS3- |
| | | | | protease (Central |
| | | | | European tick-borne |
| | | | | fever viruses and the |
| | | | | like) |
| NS3 | 68-70 kD | 70 kD | 70 kD | serine protease, helicase |
| NS4A | — | 16 kD | 8-10 kD | Membrane-associated, |
| | | | | associated with NS3- |
| | | | | protease (hepatitis C |
| | | | | virus) |
| NS4B | — | 27 kD | 27 kD | Membrane-associated |
| NS5 | 100-104 kD | 100 kD | — | RNA-capping, (non- |
| | | | | hepatitis C virus) |
| NS5A | — | — | 56-58 kD | RNA-dependent |
| NS5B | — | — | 68-70 kD | RNA-polymerase |

As has already been explained earlier, HCV has a high rate of mutation, with hypervariable and variable regions as well as relatively conserved sequence section being observed in the genome. The hypervariable regions are located in the aminoterminal region of the E2-protein between the amino acids 1-27 and 90-97. They are recognised by the antibody and, thus, are exposed to a strong immunologic selection pressure. In the course of a chronic infection, the virus changes the two epitopes in such a way that the neutralised antibodies of the human are no longer able to recognise them anymore. As a result, it is supposed that virus variants are formed which may trigger a chronic infection.

It has not been clarified entirely in which way the HCV supports the formation of a primary hepatocellular carcinoma. There are indications that specific sequences of the C-protein interact with the Ras-protein, thereby inducing the transformation (Taylor, 1993, Sem. Virol. 4: 305-312). The time between the infection and the formation of a primary hepatocellular carcinoma is approx. 40 years. In particular in Japan, double infections with the hepatitis B virus and HCV have been described in up to 18% of the primary hepatocellular carcinoma (Taylor, loc. cit.), with a simultaneous infection with both viruses reducing the time between incubation and the formation of a carcinoma.

So far, scientists have not succeeded in developing and applying a vaccine. In the case of a chronic infection, the use of interferon-αand/or ribavirin, too, has proven to be successful only in part. Many of the quasi species of HCV that are formed are resistant to an interferon therapy.

Thus, there is a great need for treating existing infections triggered by flaviviruses, in particular HCV infections, and interfering with the formation particularly of HCV infections in high-risk groups such as drug addicts or children of infected mothers in a prophylactic manner. Thus, the technical problem of the present invention was to provide ways and means for treating infections and diseases based thereon which are triggered by viruses belonging to the group of Flaviviridae, in particular HCV, and for preventing the formation of such infections.

This technical problem has been solved by providing the embodiments characterised in the claims. Thus, the invention relates to the use of one or more Phyllanthus components or substances or mixtures of substances obtained therefrom for preventing or treating infectious diseases that are triggered by viruses belonging to the family of Flaviviridae or infectious diseases in which viruses belonging to the family of Flaviviridae are involved in the development or progression thereof. The term “Phyllanthus component(s)”, as used herein according to the invention, includes all components of the whole plant such as leaves, cortex, flowerings, seeds, fruits, stalk, branches, stem, roots and wood as well as parts thereof such as leaf or root tips. These Phyllanthus components may have the same, similar or non-related substances. Therefore, various Phyllanthus components may be used alone or in combination with each other. The term “more” Phyllanthus components also includes the entirety of the Phyllanthus components, for instance in form of whole plants or the extracts thereof. The Phyllanthus components may be used after pre-treatment or without any pre-treatment. A pre-treatment may comprise, for example, steps like drying the leaves.

The term “substances and mixtures of substances” which may be obtained therefrom mainly include active agents. These may be used in a pure form or together with additional components. These active agents may be used alone or in combination as an active agent or as a mixture of active agents. In this case, different active agents may be present in different Phyllanthus components or in the same Phyllanthus components. Mixtures of substances may be obtained from the same Phyllanthus component or from different Phyllanthus components. In this case, the active agents may first be obtained individually from the different Phyllanthus components, wherein the individual active agents are then mixed to form a mixture of active agents, or a mixture of active agents may simultaneously be obtained from the Phyllanthus components which have previously been mixed. Substances or mixtures of substances of the invention may consist of the same or different members of a class of chemical compounds. These chemical compounds may be used in a natural form, i.e. without being changed by the process of recovery, or may be used in a form in which they were changed by the process of recovery. The chemical components may, for instance, be cyclical or branched or unbranched polar or non-polar hydrocarbons, lipids, lipid derivatives, nucleic acids, nucleotides, nucleotide derivatives, nucleosides, nucleoside derivatives, polypeptides, peptides, amino acids, modified amino acids, saturated and unsaturated fatty acids. The term “derivative” as used herein includes all compounds that may be derived from the compounds stated and that may be obtained by means of chemical modification. Such derivatives may, for example, be obtained by substitution, addition, esterication, saponification or condensation reactions. Of particular importance as active agents are for instance: flavonoids, organic acids and their esters, e.g. caffeic acid esters, gallotannins, polysaccharides, glycoproteins, peptides and proteins (ribosome-inactive proteins, i.e. so-called RIPs).

Within the meaning of the present invention, the term “preventing” means using Phyllanthus components or substances or mixtures of substances obtained therefrom as a prophylactic measure for preventing an infection with flaviviruses or for reducing the virus load. Furthermore, within the meaning of the invention, the term “preventing” means preventing the disease from breaking out or reducing the seriousness of the disease breaking out. The Phyllanthus components or the substances or mixtures of substances that are used for prevention may differ from or be identical with the Phyllanthus components or the substances or mixtures of substances which are used for treating the disease. The dose and application used for prevention may be different from or identical with the ones used for treatment. Prevention may be achieved by one single or several application(s). The dose of Phyllanthus components or substances or mixtures of substances obtained therefrom should, however, essentially offer sufficient protection against infectious diseases triggered by viruses belonging to the family of Flaviviridae or of infectious diseases in which viruses belonging to the family of Flaviviridae are involved in the development or progression thereof. The dose used for prevention should not be harmful to the organism.

If one or more Phyllanthus components or substances or mixtures of substances obtained therefrom are used for treating infectious diseases, these Phyllanthus components or substances or mixtures of substances obtained therefrom may be applied in a manner similar to the one described for prevention. The dose used for the treatment may fight both the cause of the infectious disease, i.e. the viruses or their propagation in the body, and the symptoms of the infectious diseases. In general, it is to be noted that the use of natural therapeutics such as Phyllanthus components or substances or mixtures of substances obtained therefrom are advantageous for both the prevention and the treatment of infectious diseases triggered by flaviviruses and diseases that are secondarily triggered by them since natural therapeutics, in particular therapeutics based on Phyllanthus components or substances or mixtures of substances obtained therefrom, due to a better tolerance, are expected to have fewer side-effects which are an additional strain to the body. Moreover, substances or mixtures of substances obtained from plants mostly have a multivalent effect, e.g. Phyllanthus has both an immunomodulatory and an antiviral activity, and an anti-oxidative and hepatoprotective effect.

For the treatment, the components or substances or mixtures of substances are formulated as a pharmaceutical composition, a medicinal product or a medicinal adjuvant. For a definition of pharmaceutical compositions, see below.

Infectious diseases are particularly characterised by the fact that an immune response is triggered in the organism caused by a pathogen. The immune response is intended to fight the pathogen. However, dangerous damage to the cells and the tissues of the organism affected may be caused, which, in this case, may be caused by the body's own immune system. Besides that, as regards infectious diseases, however, the pathogen itself may cause damage since, for instance, metabolic processes or differentiating programmes in the affected cells may be triggered which have a harmful effect on the organism. Cause and symptoms of the infectious diseases may affect identical or different cells or tissues. Thus, for instance, a changed metabolism in the liver could lead to a changed composition of the proteins contained in the blood, in particular of the enzymes, which could result in damage to other organs such as the kidney. Therefore, the use of Phyllanthus components or substances or mixtures of substances obtained therefrom according to the present invention does not only relate to the prevention or treatment of causes of infectious diseases such as an infection with viruses, but also to the prevention or treatment of secondary symptoms which are based on the presence of the pathogen in the organism.

Within the meaning of the present invention, “pathogens” are viruses belonging to the family of Flaviviridae. The family of Flaviviridae currently comprises three genera, i.e. flavivirus, hepatitis C-like virus, pestivirus, as well as a number of non-classified Flaviviridae.

As regards the genus flavivirus, the group of dengue viruses, which currently consists of dengue virus, dengue virus type 1, dengue virus type 2, dengue virus type 3 and dengue virus type 4, the group of Japanese encephalitis viruses, which currently consists of Alfuy virus, Japanese encephalitis virus, Kokoberga virus, Koutango virus, Kunjin virus, Murray Valley encephalitis virus, St. Louis encephalitis virus, Strafford virus, Usutu virus and West Nile virus, the group of modoc viruses, which currently consists of Cowbone Ridge virus, Jutiapa virus, Modoc virus, Sal Vieja virus and San Perlita virus, the group of mosquito-borne viruses, which currently consists of IIheus virus and Sepik virus, the group of Ntaya viruses, which currently consist of Bagaza virus, Israel turkey meningoencephalitis virus, Ntaya virus, Tembusu virus and Yokose virus, the group of Rio Bravo viruses, which currently consists of Apoi virus, Bukalasa bat virus, Dakar bat virus, Entebbe bat virus, Rio Bravo virus and Saboya virus, the group of tick-borne encephalitis viruses, which currently consists of Carey island virus, deer tick virus, Karshi virus, Kumlinge virus, Kyasanur forest disease virus, Langat virus, Louping ill virus, Negishi virus, Omsk hemorrhagic fever virus, Phnom Penh bat virus, Powassa virus, Royal Farm virus, Skalica virus, tick-borne encephalitis virus and Turkish sheep encephalitis virus, the group of Tyuleniy viruses, which currently consists of Meaban virus, Saumarez Reef virus, Tyuleniy virus, the group of Uganda-S viruses, which currently consists of Banzi virus, Bouboui virus, Edge Hill virus and Uganda-S virus, the group of yellow fever viruses, which currently consists of yellow fever virus, as well as a group of non-classified members of the genus flavivirus, which currently comprises Aroa virus, Batu Cave virus, Bussuquara virus, Cacipacore virus, flavivirus Central European tick-borne fever, Gadgets Gully virus, Greek goat encephalitis virus, Iguape virus, Jugra virus, Kadam virus, Kedougou virus, Montana myotis leukoencephalitis virus, Naranjal virus, Potiskum virus, Rocio virus, Russian Spring-Summer encephalitis virus, Saint Louis encephalitis virus, Sokuluk virus, Spanisch sheep encephalitis virus, Spondweni virus, tick-borne flavivirus, Yaounde virus, Zika virus and flavivirus sp. are of particular importance.

As regards the genus of hepatitis C-like viruses, the hepatitis C viruses: hepatitis C virus (isolate 1), hepatitis C virus (isolate BK), hepatitis C virus (isolate EC1), hepatitis C virus (isolate EC10), hepatitis C virus (HC-J2), hepatitis C virus (isolate HC-J5), hepatitis C virus (isolate HC-J6), hepatitis C virus (isolate HC-J7), hepatitis C virus (isolate HC-J8), hepatitis C virus (isolate HC-JT), hepatitis C virus (isolate HCT18), hepatitis C virus (isolate HCT27), hepatitis C virus (isolate HCV-476), hepatitis C virus (isolate HCV-KF), hepatitis C virus (isolate Hunan), hepatitis C virus (isolate Japanese), hepatitis C virus (isolate Taiwan), hepatitis C virus (isolate TH), hepatitis C virus isolate H, hepatitis C virus type 1, hepatitis C virus type 10, hepatitis C virus type 2, hepatitis C virus type 3, hepatitis C virus type 4, hepatitis C virus type 5 and hepatitis C virus type 6 are of particular importance.

As regards the genus of pestiviruses, the group of bovine viral diarrhea virus-2 viruses, which currently consists of border disease virus strain C413, bovine viral diarrhea virus-2 isolate 230/98-K1 (Gi-4), bovine viral diarrhea virus-2 isolate 230/98-K2 (Gi-5), bovine viral diarrhea virus-2 isolate 230/98-K3 (Gi-6), bovine viral diarrhea virus-2 isolate Giessen-3 und bovine viral diarrhea virus-2 isolate SCP, the group of pestiviruses Giraffe H138 or Strain Girafe-1, the group of the type-1 pestiviruses, which currently consists of bovine viral diarrhea virus (isolate NADL), bovine viral diarrhea virus (strain SD-1), bovine viral diarrhea virus strain 4979, bovine viral diarrhea virus strain 5.19006, bovine viral diarrhea virus strain 5.19516, bovine viral diarrhea virus strain 60.875, bovine viral diarrhea virus strain 6384, bovine viral diarrhea virus strain 7923, bovine viral diarrhea virus strain 97/123, bovine viral diarrhea virus strain 97/360, bovine viral diarrhea virus strain 97/730, bovine viral diarrhea virus strain Bovax20, bovine viral diarrhea virus strain Changchun 184, bovine viral diarrhea virus strain Cumulus, bovine viral diarrhea virus strain D, bovine viral diarrhea virus strain H, bovine viral diarrhea virus strain L1305, bovine viral diarrhea virus strain L4262, bovine viral diarrhea virus strain Oregon C24V, bovine viral diarrhea virus strain Rit 4350, bovine viral diarrhea virus strain V071094, bovine viral diarrhea virus strain V110794, bovine viral diarrhea virus strain V11295, bovine viral diarrhea virus strain 130995b, bovine viral diarrhea virus strain V190695, bovine viral diarrhea virus strain Yak, bovine viral diarrhea virus-1 and pestivirus type 1 strain R2727, the group of type-2 pestiviruses, which currently consists of Hog cholera virus, the group of type-3 pestiviruses, which currently consists of border disease virus strain 135661, border disease virus strain 137/4, border disease virus strain 170337, border disease virus strain 8320-22NZ, border disease virus strain 8320-31 NZ, border disease virus strain A1263/2, border disease virus strain A1870, border disease virus strain A841/1, border disease virus strain Cumnock, border disease virus strain D1586/2, border disease virus strain Frijters, border disease virus strain G1305, border disease virus strain G2048, border disease virus strain JH2816, border disease virus strain K1729/3, border disease virus strain L83-84, border disease virus strain L991, border disease virus strain Moredun, border disease virus strain Moredun cp, border disease virus strain Moredun ncp, border disease virus strain Q1488/1, border disease virus strain Q1488/6, border disease virus strain Q1673/2, border disease virus strain T1789/1, border disease virus strain T1802/1, border disease virus strain V-TOB, border disease virus strain V1414, border disease virus strain V2377/12, border disease virus strain V2536/2, border disease virus strain V3196/1 and border disease virus strain X818, as well as a group of non-classified members of the genus pestiviruses, which currently comprises border disease virus strain 90/8320/31, border disease virus strain 91/5809, border disease virus strain A1263/1, border disease virus strain CB2, border disease virus strain CB5, border disease virus strain Idaho207, border disease virus strain Idaho209, border disease virus strain ldaho201, bovine viral diarrhea virus-1 strain R2727, pestivirus isolates, porcine pestivirus, Pestivirus sp., Pestivirus sp. Bison-1 and Pestivirus sp. Reindeer-1 are of particular importance.

The non-classified Flaviviridae currently comprise douroucouli hepatitis GB virus A, GBV-A-like virus, the group of GBV-C/HG viruses, which currently consists of hepatitis G virus, hepatitis GB virus C, hepatitis GB virus C-like virus and hepatitis GB virus C/G (hepatitis GB virus C/hepatitis G virus), hepatitis GB virus A, hepatitis GB virus B, marmoset hepatitis GB virus A, mosquito cell fusing agent and turkey meningoencephalitis virus.

Apart from the current members listed above, the family of Flaviviridae also include all the viruses that will be discovered or assigned to the family in future. The family of Flaviviridae also include mutants of individual members. Mutants are viruses having a changed genetic information. The changed genetic information may, for example, cause resistance to therapeutics. Such a resistance may, for example, be based on the formation of mutated proteins resulting from changed genetic information. The members of the family of Flaviviridae are particularly characterised in that they all trigger dangerous infectious diseases, with hepatitis triggered by the hepatitis C virus being of particular importance resulting in changes in the morphology which are life-threatening to the organism and, at the same time, in the function of the liver. Furthermore, prophylactic measures such as the use of Phyllanthus components or substances or mixtures of substances obtained therefrom according to the invention are suggested for prevention due to the ways in which these viruses are transmitted, in particular saliva and blood. The Phyllanthus components or substances or mixtures of substances obtained therefrom according to the invention may alleviate the infectious diseases triggered by the viruses belonging to the family of Flaviviridae, in particular by inhibiting the propagation of the viruses in the cells and tissues affected. This may, for instance, be achieved by inhibiting individual or various enzymes which are responsible for the cellular changes in connection with the infectious disease. In addition, however, the Phyllanthus components or substances or mixtures of substances obtained therefrom according to the invention may, for instance, exert the function of immunomodulators and positively influence the prevention or treatment of the infectious diseases.

The term “development and progression” means that the viruses belonging to the family of Flaviviridae may, as a pathogen, either cause the infectious disease and, thus, may be involved in the development thereof either alone or in combination with other pathogens, or may support the progression of an infectious disease already existing either alone or in combination with other pathogens.

The use according to the invention shows surprising and unexpected positive results as regards the prevention or treatment of infectious diseases, in particular of those based on HCV infects.

These positive and surprising effects could not be expected based on the data have been known so far as to the effect of Phyllanthus components. However, it has been known that Phyllanthus species have an anti-bacterial effect, yet this effect depends on the bacteria species. Therefore, it is not possible to draw a conclusion as to the effects that Phyllanthus components have on bacteria species which have not been tested yet since the molecular basis of the anti-bacterial effect of Phyllantus components has not yet been known. Likewise, it is not possible to conclude a corresponding effect on viruses belonging to a completely different family from the anti-viral effectiveness of Phyllanthus in the treatment of hepatitis B infections. This is, amongst others, based on the fact that the structural proteins of the virus and the virus enzymes of hepatitis B and hepatitis C have a completely different structure. Even if one assumes the positive pharmaceutical effects against hepatitis B infections that may be achieved by applying Phyllanthus components and that are known in the state of the art, the considerably strong reduction of the virus load in patients treated is to be regarded as surprising: within less than 9 months, the HCV load in a patient suffering from a chronic HIV infection was reduced to reach almost the detection limit.

Further advantages as to the use of Phyllanthus components or substances or mixtures of substances obtained therefrom according to the invention are that, in spite of the reduction in the titre, there are no side-effects worth mentioning compared to conventional methods of treatment and that the clinical findings such as depression and, thus, inability to work were improved.

Furthermore, the invention relates to the use of one or more Phyllanthus components or substances or mixtures of substances obtained therefrom for producing a pharmaceutical composition, medicinal product or a medicinal adjuvant for preventing or treating infectious diseases that are triggered by viruses belonging to the family of Flaviviridae or of infectious diseases in which viruses belonging to the family of Flaviviridae are involved in the development or progression thereof.

According to the invention, pharmaceutical compositions are substances and preparations which are meant to heal, alleviate, prevent or recognise diseases, ailments, injuries to the body or pathologic troubles by applying said compositions to or in the human body.

According to the invention, medicinal products are all substances and preparations of substances used alone or in combination with each other or other means which are meant by the manufacturer to be used in humans by means of their function for recognising, preventing, monitoring, treating or alleviating diseases and whose intended main effect in or on the human body is achieved neither by agents having a pharmacological or immunological effect nor by metabolism, however whose effect may be supported by such agents.

According to the invention, medicinal adjuvant are substances used (as active ingredients) for the production of pharmaceutical preparations.

The essential terms of this embodiment have already been explained and defined above. The pharmaceutical compositions containing the Phyllanthus components or substances or mixtures of substances obtained therefrom is optionally formulated in combination with a pharmaceutically active carrier and/or diluent.

The person skilled in the art knows suitable pharmaceutically acceptable carriers which comprise phosphate-buffered saline solutions, water, emulsions such as oil/water emulsions, various kinds of detergents, sterile solutions, etc. Pharmaceutical compositions comprising such carriers may be formulated using conventional methods. These pharmaceutical compositions may be administered to an individual in a suitable dose. They may be administered in various ways, e.g. orally, intravenously, intraperitoneally, subcutaneously, intramuscularly, locally or intradermally. The doctor in charge will determine the kind of dosage in accordance with the clinical factors. The person skilled in the art knows that the kind of dosage depends on various factors such as the patient's size, body surface, age, sex or general health, but also on the specific agent that is administered, the duration and kind of administration and on other medicaments which may possibly be administered at the same time.

In a preferred embodiment of the use according to the invention, the infectious disease is yellow fever, dengue fever, Central European tick-borne fever, cirrhosis, hepatocellular carcinoma or membranoproliferative glomerulonephritis.

As regards the characteristic symptoms of the diseases listed, reference is made to standard works such as Pschyrembel. The infectious diseases listed or their symptoms may be directly caused by the infection or occur as a consequence thereof.

The invention also relates to the use of one or more of the Phyllanthus components or substances or mixtures of substances obtained therefrom for inhibiting the propagation of viruses belonging to the family of Flaviviridae.

The term “inhibiting the propagation of viruses belonging to the family of Flaviviridae” means that the replication of the viruses in the affected host cell or the budding of the viruses from an affected host cell or both is prevented. Furthermore, the viruses could be prevented from infecting of other host cells and from propagating by blocking the target cells (host cells). As regards the inhibition of a new formation of viruses in an affected host cell, mainly enzymes which are involved in the new formation of the viruses may be inhibited. These are, in particular, enzymes which are involved in replicating viral nucleic acids (polymerases and helicases) or in processing viral proteins (proteases). Inhibition of the propagation by inhibiting the proliferation of viruses from an infected host cell could particularly be achieved by inhibiting the viral and/or cellular protein. Inhibition of the propagation of the blocking of further host cells could be achieved by inhibiting the mechanisms involved in the recognition of host cells and virus or in the take-up of the virus into the host cell or by inhibition.

In a preferred embodiment of the use according to the invention, the inhibition takes place in vivo, ex vivo or in vitro. This embodiment is of particular importance as regards the further development of active agents according to the invention, the analysis of new active agents or the control of the quality of the same. In particular, inhibition may also be carried out outside the organism in isolated cells or tissues, e.g. in cell culture. It may be analysed in particular in in-vitro systems, e.g. in cell cultures, which components have the most favourable characteristics of Phyllanthus. In this way, substances or mixtures of substances may be optimised on the basis if in-vitro test systems. Preferably, Phyllanthus components are divided into groups of active agents or into individual components using chemical/physical methods and the individual components are then examined for inhibition of the virus propagation in such test systems.

In another preferred embodiment of the use according to the invention, the one or more Phyllanthus components or the substances or mixtures of substances obtained therefrom are derived from Phyllanthus amarus, Phyllanthus niruri, Phyllanthus emblica, Phyllanthus urinaria, Phyllanthus acidus, Phyllanthus acuminatus and Phyllanthus reticulatus or of more of these Phyllanthus species.

In another preferred embodiment of the use according to the invention, the viruses of the family of Flaviviridae belong to the genus flavivirus.

In a most preferred embodiment of the use according to the invention, the genus flavivirus comprises the group of dengue viruses, the Japanese encephalitis viruses, the modoc viruses, the mosquito-borne viruses, the Ntaya viruses, the Rio Bravo viruses, the tick-borne encephalitis viruses, the Tyuleniy viruses, the Uganda-S viruses, the yellow fever viruses and the non-classified flaviviruses, in particular the Central European tick-borne fever flavivirus.

In another preferred embodiment of the use according to the invention, the viruses of the family of Flaviviridae belong to the genus of hepatitis C-like viruses. In a most preferred embodiment of the use according to the invention, the genus of hepatitis C-like viruses comprises the hepatitis C virus.

In another preferred embodiment of the use according to the invention, the viruses of the family of Flaviviridae belong to the genus of pestivirus.

In a most preferred embodiment of the use according to the invention, the genus of pestivirus comprises the bovine viral diarrhea virus-2, the pestivirus giraffe H138, the pestivirus strain giraffe-1, the pestiviurs type 1, the pestivirus type 2, in particualr the hog cholera virus, and the pestivirus type 3.

In another preferred embodiment of the use according to the invention, the viruses of the family of Flaviviridae belong to the genus of non-classified flaviviruses.

In a most preferred embodiment of the use according to the invention, the non-classified flaviviruses comprise the douroucouli hepatitis GB virus A, the GBV-A-like virus, the group of GBV-C/HG viruses, in particular the hepatitis G virus, the hepatitis GB virus A, the hepatitis GB virus B, the marmoset hepatitis GB virus A, the mosquito cell fusing agent and the Turkey meningoencephalitis virus.

In another preferred embodiment of the use according to the invention, the term “prevention or treatment” comprises the inhibition of one or more viral enzymes.

The term “inhibition of one or more viral enzymes” means that the function of these enzymes is either directly or indirectly influenced in a negative manner. A direct inhibition, for example, (neither in this context in any other explanations do the inventors want to be bound to one scientific therapy) may be the interaction of one or inhibiting substances contained in the Phyllanthus components or the substances or mixtures of substances obtained therefrom with one or more enzymes. An indirect inhibition may be the interaction of said inhibitors with factors that are necessary either for expressing, translating or processing said enzymes. In this case, inhibition in particular means the inhibition of viral enzymes such as helicases, polymerases or proteases. A direct inhibition of said enzymes could, for example, be based on the interaction of inhibitors contained in Phyllanthus components or substances or mixtures of substances obtained therefrom with the catalytic centre of said enzymes or with a domain which regulates the activity. Furthermore, other cellular enzymes, which, for instance, participate in the expression, translation or post-translational modification of the viral enzymes and which, thus, regulate their function, may be affected or additionally affected by said inhibitors. In any case, however, the propagation and/or the infectious disease resulting from the infection with the virus or the development or progression of such a disease is reduced or alleviated by the inhibition according to the invention of one or more viral enzymes.

In a particularly preferred embodiment of the use according to the invention, the inhibition comprises inhibiting the processing of one or more viral enzymes.

The post-translational modification of viral enzymes by Phyllanthus components or substances or mixtures of substances obtained therefrom, which has been described earlier, it is mainly the processing of one or more viral enzymes that is of importance. The term “processing” means that an enzymes which is inactive at first is activated by post-translational modifications, e.g. proteolytic cleavage, phosphorylation or glycosylation. Therefore, the function of said enzymes may be harmed or inhibited completely by inhibiting, according to the invention, the processing of one or more viral enzymes. The Phyllanthus components or substances or mixtures of substances obtained therefrom according to the invention which may be used for inhibiting the processing, may inhibit one or more viral enzymes at the same time. This can be achieved by inhibiting the same or different processing steps that are necessary for the activation of the enzyme.

In another particularly preferred embodiment of the use according to the invention, the viral enzymes are selected from the group consisting of NS2A, NS2B, NS3, NS4A, NS5, NS5A and NS5B.

The enzymes NS2A, NS2B, NS3 and NS4A belong to the class of viral proteases and/or helicases. The enzymes NS5, NS5A and/or NS5B belong to the family of viral polymerases.

In another preferred embodiment of the use according to the invention, the Phyllanthus components comprise herba drug, leaves, cortex, flowering, seeds, fruits, stalk, branches, stem, roots and wood.

In another preferred embodiment, the substances and mixtures of substances are selected from the group consisting of alkaloids, tannins, gallotannins, lignans, sesquiterpenes, triterpenes, proteins, peptides, polysaccharides, glycoproteins, flavonoids, sterols and caffeic acid esters.

Alkaloids are a group of substances which is characterised in that it contains one or more nitrogen atoms, is present as a heterocycle and generally is bound to a plant acid in a saline manner. Chemically, a differentiation can be made between protoalkaloids, also comprising biogenic amines as alkaloid precursors, and pseudo-alkaloids. In a narrower sense, alkaloids are descendants of specific acid families such as presecurines. Tannins are particularly characterised by phenolic hydroxyl groups. The group of lignans is chemically characterised by phenylpropane condensate compounds (cyclic, open-chain). The group of terpenes, which comprises sesquiterpenes and triterpenes, consist of unsaturated hydrocarbons formed by isoprene basic units and are present as a polymers. The substances and mixtures of substances according to the invention may be present as a pure substance or may have additional components. Moreover, the mixtures of substances may consist of different members of said group and may, in addition, contain further substances or mixtures of substances.

In another preferred embodiment of the use according to the invention, alcohols and/or a mixture thereof are used for extracting the substance or the mixture of substances.

The term “alcohols” means, in particular, hydrocarbons which have one or more OH-groups having a detergent or amphoteric effect at their carbon atoms. Particularly alcohols having a structure of 1 to 4 carbon atoms and/or mixtures thereof with methanol and ethanol mixtures being of particular importance.

In a particularly preferred embodiment of the use according to the invention, the alcohols are short-chain primary C1 to C4 alcohols and/or mixtures thereof.

In the most preferred embodiment of the use according to the invention, the alcohols are methanol and/or ethanol.

In another preferred embodiment of the use according to the invention, one or more Phyllanthus components or the substances or mixtures of substances obtained therefrom may be used in combination with one or more other active agents.

The Phyllanthus components according to the invention or the substances or mixtures of substances obtained therefrom may also be used in combination, i.e. directly as a mixture, contemporaneously or subsequently with one or more other active agents. The term “other active agents” means, in particular, pharmaceutical compositions, alone or a combination thereof, which are used for treating infectious diseases triggered by a member of the family of Flaviviridae.

In particular cytokines such as immunomodulators, e.g. interferon α, interferon β, interferon γ or interleukins, nucleoside analogues such as ribavirin, adefovir dipivoxil, famciclovir or FTC as well as antibodies, in particular monoclonal antibodies have proven to be effective active agents for treating or preventing infectious diseases based on a member of the family of Flaviviridae. The effect of interferons in mainly based on the modulating effects they have on the cells of the immune system, whereas the effect of ribavirin mainly is the inhibition of enzymes. A therapy combining two or more of said active agents should have a particularly positive effect on both the prevention and the therapy of the disease.

In a particularly preferred embodiment of the use according to the invention, the other active agents are selected from the group of cytokines.

In a most preferred embodiment of the use according to the invention, the group of cytokines comprises immune modulators, in particular interferon α, interferon β, ingterferon γ and interleukins.

In another preferred embodiment of the use according to the invention, the other active agents are selected from the group of nucleoside analogues.

In a most preferred embodiment of the use according to the invention, the group of nucleoside analogues comprises ribavirin, adefovir dipivoxil, famciclovir and FTC.

In another preferred embodiment of the use according to the invention, the other active agents are selected from the group of antibodies. In another preferred embodiment of the use according to the invention, the application is in form of an infusion solution, injection solution, oral form of application, ointment, therapeutic pack, a granulate, food supplement or in form of clysters.

Phyllanthus components or substances or mixtures of substances obtained therefrom may be used in different forms either separately or in combination with each other. In particular, the Phyllanthus components or substances or mixtures of substances obtained therefrom according to the invention may be applied in form of infusion solutions or injection solutions. In this case, active agents isolated from Phyllanthus components or substances or mixtures of substances obtained therefrom are mixed, either purely or mixed with additional components, with a pharmaceutically acceptable solvent and injected into the blood vessel of a patient who suffers from an infectious disease based on the infection with Flaviviridae. Therefore, in therapy, infusion solutions and injection solutions are preferably suitable form of application. Oral forms of application are, for example, a powdery or granulated mixture of active agents and adjuvant having regular shapes that are always the same (e.g. in the shape of a disc, cylinder, egg, ball, rod or cube) so that a uniform dosage is guaranteed. The active agents may be isolated from the Phyllanthus components or substances or mixtures of substances obtained therefrom in pure form or mixed with other components. The adjuvant are used particularly for rendering the composition pharmaceutically acceptable and for conserving the active agents and for making them. storable. In another embodiment, the active agents are administered in an oil-containing liquid in a capsule. An ointment having an active agent which may be present in pure form or mixed with additional components and which may be prepared from Phyllanthus components or substances or mixtures of substances obtained therefrom, is a pharmaceutical composition which can be plastically formed and which, apart from the active agent, comprises an ointment basis as a basis or a vehicle. Furthermore, the active agents may, for instance, be administered as emulsions. Therapeutic packs which may, for instance, be applied to the skin can, in particular either contain the Phyllanthus components as such or the substances or mixtures of substances obtained therefrom in pure form or mixed with additional components and comprise the active agents isolated from the Phyllanthus components or the substances or mixtures of substances obtained therefrom in pure form or mixed with additional components in combination with carrier material as well as further active agents. A granulate is a coarse-grained, granulate-containing powder mixture containing active agents of the Phyllanthus components or the substances or mixtures of substances obtained therefrom according to the invention. A granulate may also have an oral form of application. The Phyllanthus components or the substances or mixtures of substances obtained therefrom according to the invention may also be used as food supplements. Apart from using Phyllanthus components as foodstuffs, also in particular food supplements, e.g. in form of oral forms of application, granulates or therapeutic teas.

In another preferred embodiment of the use according to the invention, the application is orally, topically or parenterally.

Furthermore, the invention relates to a method for preventing or treating infectious diseases in a mammal that are triggered by viruses belonging to the family of Flaviviridae or of infectious diseases in which viruses belonging to the family of Flaviviridae are involved in the development or progression thereof, wherein the mammal is administered one or more Phyllanthus components or substances or mixtures of substances obtained therefrom.

Apart from administering one or more Phyllanthus components or substances or mixtures of substances obtained therefrom, the method of the invention may also comprise further steps such as the administration of other therapeutics or additional therapeutic measures. The definitions according to the uses of the invention also apply to the methods of the invention or the terms used in the specification. Moreover, the methods of the invention also comprise all the embodiments of the uses according to the invention.

In another preferred embodiment of the method according to the invention, the mammal is a human.

In addition, the invention relates to a method for inhibiting the propagation of viruses belonging to the family of Flaviviridae, wherein one or more Phyllanthus components or substances or mixtures of substances obtained therefrom is contacted with the viruses.

In a preferred embodiment of the method according to the invention, the inhibition takes place in vivo, ex vivo or in vitro.

In another preferred embodiment of the method according to the invention, the virus is the hepatitis C virus (HCV).

The other preferred or particularly preferred embodiments described with regard to the use according to the invention apply mutatis mutandis to the methods of the invention.

The examples illustrate the invention.

### EXAMPLE (1)

Use of Phyllanthus Components for Treating a Chronic HCV Infection

On Nov. 3, 1995, a 34-year old male patient presented himself with a chronic HCV infection. The HCV infection had already been diagnosed in January 1995. According to the Mori classification of HCV, genotype 3a had been diagnosed. The patient suffered from tiredness, reduced appetite and reduced endurance. Furthermore, he had an enlarged liver. Since it was known that the patient had been addicted to drugs until January 1995, it was assumed that he got infected by using contaminated injections and/or needles.

From May 1996 until Jan. 29, 1997, the patient was treated with herba Phyllanthus amarus (dried plant powder) in a daily dose of 2250 mg/d. Then, the dose was increased to 3000 mg/d. Moreover, from Oct. 14, 1996 until December 1997, the therapy was supplemented by Liv-52.

During the therapy, the tiredness disappeared and the appetite and endurance increased significantly. All in all, the quality of the patient's life improved considerably. The load of viral RNA in the body fell from 8.09×10⁶ copies/ml at the beginning of the therapy to 0.14×10⁶ copies/ml at the end of the therapy. The reduction of the number of copies of HCV RNA is shown in the table below.

TABLE 2

| Date of examinination | HCV RNA (10⁶ copies/ml) |
|---|---|
| 22/08/96 | 8.09 by means of bDNA technique¹ (detection |
| | limit 0.20 Meq/ml) |
| 14/10/96 | 6.50 by means of bDNA technique |
| 29/01/97 | 0.47 by means of qPCR² (detection limit 300 |
| | copies/ml) |
| 06/05/97 | 0.14 by means of qPCR |

¹The term “bDNA” stands for branched DNA which is described e.g. in Komurian-Pradel et al. (2001), J Virol Methods, 95, 111-119
²The term “qPCR” stands for quantitative PCR which is described e.g. in Kearns et al. (2001) J Virol Methods, 95, 121-131

In patients that are chronically infected with HCV, normally stable and constant amounts of RNA can be detected over a long period of time (Gordon 1998, Hepatology 28 (6): 1702-1796). High amounts of HCV RNA are considered a negative indication of a successful therapy with interferon α and ribavirin (Weiland 1999, J. of Hepatology 31: 168-173). The patient described herein had large amounts of HCV RNA before treatment with Phyllanthus amarus so that it was possible to show that Phyllanthus amarus directly influences the replication of HCV. During the therapy, the HCV RNA load in the body was reduced drastically and persisted after that on a low level. In addition, the serologic reduction of the viral load correlated with an improvement in the patient's quality of life.

### EXAMPLE 2

In-vitro Detection of the effect of Phyllanthus Components

KL-2 bovine lung cells were plated in micro-titre plates at a density of 1×10⁵/ml. After 24 hours, the cells are infected with BVDV at a m.o.i. of 0.01. 30 minutes after the infection, the substances are added at final concentrations of, e.g. 1:100, 1:1000 and 1:10000 (mixture always in triplicate). The final step is examining each individual culture for virus-induced cytopathogenic effect (CPE). A substance is regarded as antiviral if there are neither cytotoxic nor virus-induced cytopathogenic effects in the culture. Uninfected cultures serve as a negative control, a titration series of BVDV serves as a positive control.

1. Use of one or more Phyllanthus components or substances or mixtures of substances obtained therefrom for preventing or treating infectious diseases that are triggered by viruses belonging to the family of Flaviviridae or infectious diseases in which viruses belonging to the family of Flaviviridae are involved in the development or progression thereof.

2. Use of one or more Phyllanthus components or substances or mixtures of substances obtained therefrom for producing a pharmaceutical composition, medicinal preparation or medicinal adjuvant for preventing or treating infectious diseases that are triggered by viruses belonging to the family of Flaviviridae or infectious diseases in which viruses belonging to the family of Flaviviridae are involved in the development or progression thereof.

3. The use according to claim 1 or 2, wherein the infectious diseases comprise yellow fever, dengue fever, Central European tick-borne fever, cirrhosis, hepatocellular carcinoma, membranoproliferative glomerulonephritis.

4. Use of one or more Phyllanthus components or substances or mixtures of substances obtained therefrom for inhibiting the propagation of viruses belonging to the family of Flaviviridae.

5. The use according to claim 4, wherein the inhibition takes place in vivo, ex vivo or in vitro.

6. The use according to any one of claims 1 to 5, wherein the one or more Phyllanthus components or substances or mixtures of substances obtained therefrom are derived from Phyllanthus amarus, Phyllanthus niruri, Phyllanthus emblica, Phyllanthus urinaria, Phyllanthus acidus, Phyllanthus acuminatus and Phyllanthus reticulatus or from several of these Phyllanthus species.

7. The use according to any one of claims 1 to 6, wherein the viruses of the family of Flaviviridae belong to the genus flavivirus.

8. The use according to claim 7, wherein the genus flavivirus comprises the group of dengue viruses, the Japanese encephalitis viruses, the modoc viruses, the mosquito-borne viruses, the Ntaya viruses, the Rio Bravo viruses, the tick-borne viruses, the Tyuleniy viruses, the Uganda-S viruses, the yellow fever viruses and the non-classified flaviviruses, in particular the Central European tick-borne fever flavivirus.

9. The use according to any one of claims 1 to 6, wherein the viruses of the family of Flaviviridae belong to the genus of hepatitis C-like viruses.

10. The use according to claim 9, wherein the genus of hepatitis C-like viruses comprises the hepatitis C virus.

11. The use according to any one of claims 1 to 6, wherein the viruses of the family of Flaviviridae belong to the genus of pestivirus.

12. The use according to claim 11, wherein the genus of pestivirus comprises the group of bovine viral diarrhea virus-2, the pestivirus giraffe H138, the pestiviruses strain giraffe-1, the pestivirus type 1, the pestivirus type 2, in particular the hog cholera virus, and the pestivirus type 3.

13. The use according to any one of claims 1 to 6, wherein the viruses of the family of Flaviviridae belong to the non-classified flaviviruses.

14. The use according to claim 13, wherein the non-classified flaviviruses comprise douroucouli hepatitis GB virus A, GBV-A-like virus, the group of GBV-C/HG viruses, in particular the hepatitis G virus, hepatitis GB virus A, hepatitis GB virus B, the marmoset hepatitis GB virus A, mosquito cell fusing agent and turkey meningoencephalitis virus.

15. The use according to any one of claims 1 to 14, wherein the prevention or treatment comprises the inhibition of one or more viral enzymes.

16. The use according to claim 15, wherein the inhibition comprises the inhibition of the processing of one or more viral enzymes.

17. The use according to claim 15 or 16, wherein the viral enzymes are selected from the group consisting of NS2A, NS2B, NS3, NS4A, NS5, NS5A and NS5B.

18. The use according to any one of claims 1 to 17, wherein the Phyllanthus components comprise, herba drug, leaves, cortex, flowering, seeds, fruits, stalk, branches, stem, roots and wood.

19. The use according to any one of claims 1 to 18, wherein the substances or mixtures of substances are selected from the group consisting of alkaloids, tannins, gallotannins, lignans, sesquiterpenes, triterpenes, proteins, peptides, polysaccharides, glycosides, glycoproteins, flavonoids, sterols and caffeic acid esters.

20. The use according to claim 19, wherein alcohols and/or mixtures thereof are used for extracting the substances or mixtures of substances.

21. The use according to claim 20, wherein the alcohols used are short-chain primary C1 to C4 alcohols and/or mixtures thereof.

22. The use according to claim 21, wherein the alcohols used are methanol and/or ethanol.

23. The use according to any one of claims 1 to 22, wherein one or more of the Phyllanthus components or the substances or mixtures or substances obtained therefrom are used in combination with one or more other active agents.

24. The use according to claim 23, wherein the other active agents are selected from the group of cytokines.

25. The use according to claim 24, wherein the group of cytokines comprises immune modulators, in particular interferon α, interferon β, interferon γ and interleukins.

26. The use according to claim 23, wherein the other active agents are selected from the group of the nucleoside analogues.

27. The use according to claim 26, wherein the group of the nucleoside analogues comprises ribavirin, adefovir dipivoxir, famciclovir and FTC.

28. The use according to claim 23, wherein the other active agents are selected from the group of antibodies.

29. The use according to any one of claims 1 to 28, wherein the application is in form of an infusion solution, an injection solution, oral forms of application, an ointment, a therapeutic pack, a granulate, food supplements or in form of clysters.

30. The use according to any one of claims 1 to 29, wherein the application takes place orally, topically or parenterally.

31. A method for preventing or treating infectious diseases in a mammal that are triggered by viruses belonging to the family of Flaviviridae or infectious diseases in which viruses belonging to the family of Flaviviridae are involved in the development or progression thereof, wherein one or more Phyllanthus components or substances or mixtures of substances obtained therefrom are administered to the mammal.

32. The method according to claim 31, wherein the mammal is a human.

33. A method for inhibiting the propagation of viruses belonging to the family of Flaviviridae, wherein one or more Phyllanthus components or substances or mixtures of substances obtained therefrom are contacted with the viruses.

34. The method according to claim 33, wherein the inhibition takes place in vivo, ex vivo or in vitro.

35. The method according to any one of claims 31 to 34, wherein the virus is the hepatitis C virus (HCV).
